(19) United States
(12) Patent Application Publication
(10) Pub. No.: US 2009/0312778 A1
(43) Pub. Date: Dec. 17, 2009

(54) APPARATUS FOR REMOVING PLAQUE FROM AN ARTERIAL WALL AND METHOD OF USE THEREFOR

(75) Inventor: Cargill H. ALLEYNE, Martinez, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: MEDICAL COLLEGE OF GEORGIA RESEARCH INSTITUTE, INC., Augusta, GA (US)

(21) Appl. No.: 12/484,739

(22) Filed: Jun. 15, 2009

Related U.S. Application Data
(60) Provisional application No. 61/132,130, filed on Jun. 16, 2008.

(51) Int. Cl. A61B 17/22

(52) U.S. Cl. 606/159

(57) ABSTRACT

A surgical extractor including an elongated body having a handle and working tool coupled thereto. The extractor also includes at least one user operated button and at least one blade mechanically linked to the button. The blade is selectively movable between a retracted position and a deployed position. The blade is substantially contained within the body when in the retracted position, and it is substantially projected beyond the body when in the deployed position.

### CROSS-REFERENCE TO RELATED APPLICATION

This application claims the benefit of U.S. Provisional Patent Application Ser. No. 61/132,130, filed Jun. 16, 2008, the entirety of which is hereby incorporated herein by reference.

### TECHNICAL FIELD

The present invention relates generally to the field of surgical instruments, and more particularly, to surgical instruments specializing in removing plaque from arteries.

### BACKGROUND OF THE INVENTION

Atherosclerosis causes plaque to form inside the arterial walls. For example, plaque can form inside the carotid artery, usually at the fork where the common carotid artery divides into the internal and external carotid artery. Plaque can build up in the inner surface of the artery, thereby narrowing or constricting the artery. Pieces of the plaque can break off, form emboli, and travel up the internal carotid artery to the brain, where it can block circulation and can cause death of the brain tissue.

In what is known as an endarterectomy procedure, a surgeon opens the artery, which is typically the carotid artery, and removes the plaque in an attempt to prevent complications from plaque build up. Typically, the surgeon uses multiple tools to open the carotid artery and extract the plaque build up on the carotid artery wall. For example, the surgeon often utilizes a scalpel or knife to cut open the carotid artery and then uses forceps in one hand to grasp the plaque while using a scalpel, extractor, or other instrument in the other hand to pry the plaque away from the arterial wall. Once the plaque has been at least partially separated from the arterial wall, the surgeon typically uses a pair of scissors or scalpel to cut the remaining plaque therefrom.

### SUMMARY OF THE INVENTION

The present invention relates to a surgical extractor. In one embodiment, the surgical extractor includes an elongated body having a handle and a working tool coupled thereto, at least one user operated button, and at least one blade mechanically linked to the button. The blade is selectively movable between a retracted position, wherein the blade is substantially contained within the body, and a deployed position, wherein the blade is substantially projected beyond the body.

In another form, the present invention provides an apparatus for removing plaque from an arterial wall. The apparatus includes an elongated body and a working end, wherein the working end includes a blade coupled to a dissector. Preferably, the blade is retractable within the elongated body.

In another form, the present invention provides a method for removing plaque from an arterial wall. The method includes the steps of creating an incision the arterial wall and inserting an extractor tool into the artery through the incision. The extractor tool includes an elongated body having a handle and a working tool coupled thereto, at least one user operated button, and at least one blade mechanically linked to the button, wherein the blade is selectively movable between a retracted position (where the blade is substantially contained within the body) and a deployed position (where the blade is substantially projected beyond the body). The method further includes the steps of manipulating the extractor tool to cause the working tool to separate plaque from the arterial wall; manipulating the user operated button to urge the blade to its deployed position; manipulating the extractor tool to cause the blade to cut the plaque; and removing the plaque from the arterial wall.

### BRIEF DESCRIPTION OF THE DRAWINGS

FIG. 1 depicts a side view an apparatus for removing plaque from an arterial wall according to an example embodiment of the present invention.

FIG. 2 depicts the apparatus of FIG. 1 and showing a blade in a partially deployed position.

FIG. 3 depicts a front view of the apparatus of FIG. 1.

FIG. 4 depicts the side view of the apparatus of FIG. 2 and shown with a portion of its housing removed.

FIG. 5 depicts a perspective view of a working end portion of the apparatus of FIG. 1.

FIG. 6 depicts an enlarged view of the working end portion of FIG. 4.

FIG. 7 depicts a side view of the working end portion of the apparatus of FIG. 1 and showing the blade in a fully deployed position.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

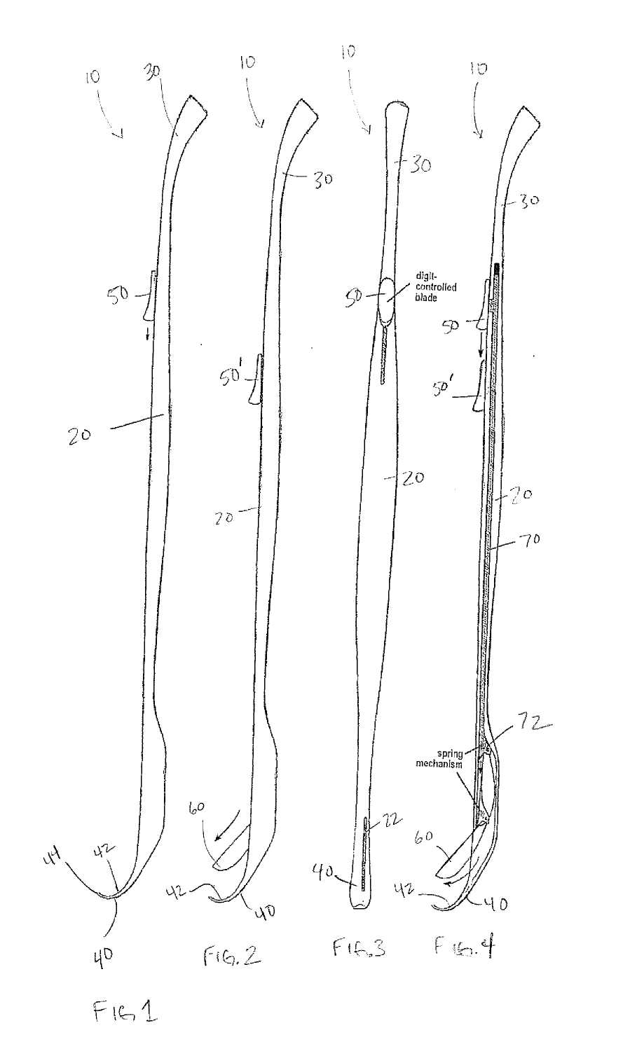

With reference now to the drawing figures, FIGS. 1-7 depict an apparatus or extractor 10 for removing plaque from an arterial wall according to an example embodiment of the present invention. Generally, the extractor 10 of the present invention can be used by a surgeon or other practitioner to remove plaque from an arterial wall by selectively prying and/or cutting it therefrom without having to use multiple tools. While the extractor of the present application is largely discussed herein as being applicable to endarterectomy procedures to remove plaque from the carotid artery, nothing herein is intended to limit the present invention to such. If fact, the extractor 10 can be used to remove plaque from other blood vessels of the body and be utilized in numerous other surgical procedures as desired by a surgeon or other practitioner.

As depicted in FIGS. 1-4, the extractor 10 generally includes an elongated body or shaft 20 having a handle portion 30 at a proximal end thereof and a working tool 40 at a distal end thereof. The working tool 40 can be shaped as desired, but in preferred embodiments, the tool has a curved tool surface 42 and a pointed tip or dissector 44 as shown more clearly in FIGS. 5-7. A curved tool surface 42 allows the surgeon to scoop or pry the plaque from an artery having an annular shaped wall.

Additionally, in example embodiments, the extractor 10 includes a mechanism for selectively retracting or deploying a blade 60 from within the body 20 to outside the body as seen in the drawing figures. In the depicted embodiment, an opening or slot 22, through which the blade 60 can be deployed, is formed in the body 20 proximate the working tool 40. In the depicted embodiment, the slot 22 is laterally offset from the center of the body 20, as shown in FIGS. 5 and 7, although in an alternative embodiment, the slot 22 can be located generally along a center line of the extractor 10. The blade 60 includes a sharp forward or free edge 62 and a generally blunt, curved rear edge 64. The curved rear edge 64 has a curvature substantially similar to that of the tool surface 42 so that when the blade 60 is fully deployed, the rear edge 64 engages and abuts the tool surface 42.

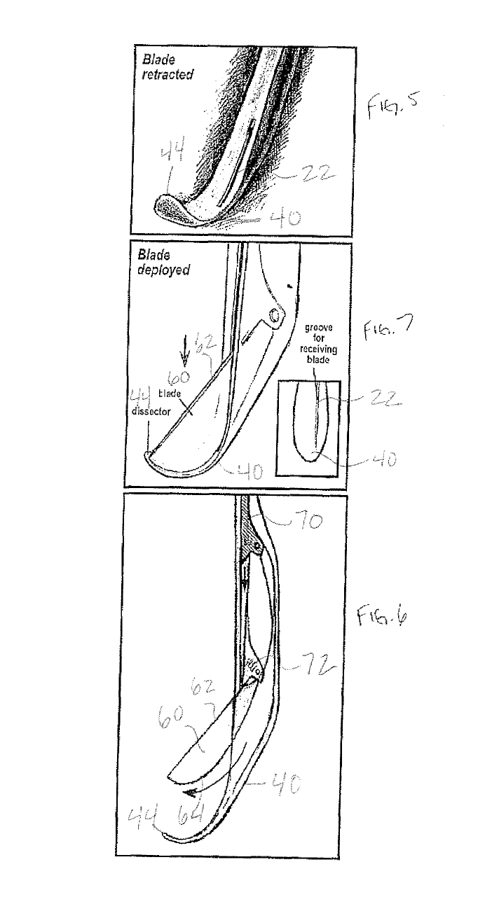

The mechanism for retracting or deploying a blade 60 includes a user operated finger button or slide 50, preferably located on or near the handle 30, for operating the action of the blade 60. Alternatively, the button 50 can be located at any place along the elongated body 20. Between the button 50 and the blade 60 are one or more linkages 70 coupled thereto. The button 50 can be slidable along a portion of the elongated body 20 such that when the button is slid toward the working tool 40, the button causes the one or more linkages 70 to drive the blade 60 out of the body 20 through the opening 22. The linkages 70 can include one or more spring biased couplings 72 to urge the blade towards the tip of the working tool 40. In preferred embodiments, the one or more linkages 70 and one or more spring biased couplings 72 are hidden within the body 20 of the extractor 10 (as shown in FIG. 4); however in alternative embodiments, the one or more linkages can be routed between the user operated button 50 and the blade 60 along an external surface of the body 20.

In example embodiments, the elements of the extractor 10 are constructed from stainless steel, but in alternate embodiments, they can be constructed from other metals (such as titanium) or metal alloys, biocompatible materials, polymers, rubbers, or other conventionally known materials.

In an alternative embodiment, the extractor can include two blades coupled together such that the sharp or cutting edges can slide past each other. In other words, the pair of blades functions in a manner similar to scissors. In such an embodiment, a first user operated button coupled to one or more mechanical linkages can deploy and retract the pair of blades, and a second user operated button coupled to one or more mechanical linkages can control the cutting action of the pair of blades. Alternatively, a single user operated button coupled to one or more mechanical linkages can be used to both deploy/retract the blades and to control the cutting action of the blades.

In use, the extractor 10 of the present invention allows a surgeon or other practitioner to manipulate the working tool 40 of the extractor 10 to lift or pry plaque away from the wall of the carotid artery. In an example method of use, the practitioner incises the arterial wall and inserts the extractor 10 into the artery through the incision. The practitioner manipulates the extractor 10 to cause the working tool 40 to separate plaque from the arterial wall. Once the practitioner has begun to scrape/pry the plaque away from the wall of the artery, he can selectively manipulate the button 50 to drive the blade 60 from an inactive or retracted position (within the body 20 of the extractor 10) to an active or deployed position (external to the body of the extractor) as depicted in FIGS. 2, 4, 6 and 7. As best seen in FIG. 4, the practitioner can manipulate the button 50 from a first position to a second position 50′, thereby forcing the blade 60 from the retracted position (FIG. 5), through the opening 22 in the extractor 10 (FIG. 6), and towards the deployed position (FIG. 7). User manipulation of the button 50 towards the working tool 40 causes the one or more linkages 70 to drive the blade 60 out of the opening 22. The practitioner can manipulate the extractor 10 to cause the blade 60 to cut the plaque and then remove the cut plaque from the arterial wall.

While the invention has been described with reference to preferred and example embodiments, it will be understood by those skilled in the art that a variety of modifications, additions and deletions are within the scope of the invention, as defined by the following claims.

What is claimed is:

1. A surgical extractor comprising:
an elongated body having a handle and a working tool coupled thereto;
at least one user operated button; and
at least one blade mechanically linked to the button;
wherein the blade is selectively movable between a retracted position and a deployed position, wherein the blade is substantially contained within the body in the retracted position, and wherein the blade is substantially projected beyond the body in the deployed position.

2. The surgical extractor of claim 1, wherein the user operated button is slidable along a portion of the elongated body.

3. The surgical extractor of claim 1, wherein the blade has a sharp free edge when in the deployed position.

4. The surgical extractor of claim 1, wherein the blade is mechanically linked to the button with one or more linkages.

5. The surgical extractor of claim 4, wherein the one or more linkages comprise one or more spring biased couplings.

6. The surgical extractor of claim 1, wherein the working tool comprises a dissector.

7. The surgical extractor of claim 1, wherein the working tool comprises a curved tool surface.

8. An apparatus for removing plaque from an arterial wall, comprising:
an elongated body; and
a working end, wherein the working end includes a blade coupled to a dissector.

9. The apparatus of claim 8, wherein the blade is retractable within the elongated body.

10. The apparatus of claim 9, wherein the blade is coupled to a user operated button for selectively deploying and retracting the blade within the elongated body.

11. The apparatus of claim 10, wherein the blade and the button are coupled together with one or more mechanical links.

12. The apparatus of claim 11, wherein the one or more mechanical links include one or more spring biased couplings.

13. The apparatus of claim 8, wherein the working end includes a curved tool surface.

14. The apparatus of claim 13, wherein a rear edge of the blade abuts the curved tool surface and wherein a free edge of the blade is sharp.

15. A method of removing plaque from an arterial wall, comprising:
creating an incision in the arterial wall;
inserting an extractor tool through the incision in the arterial wall, wherein the extractor tool includes
an elongated body having a handle and a working tool coupled thereto;
at least one user operated button; and
at least one blade mechanically linked to the button, wherein the blade is selectively movable between a retracted position and a deployed position, wherein the blade is substantially contained within the body in the retracted position, and wherein the blade is substantially projected beyond the body in the deployed position;
manipulating the extractor tool to cause the working tool to separate the plaque from the arterial wall;
manipulating the user operated button to urge the blade to the deployed position;
manipulating the extractor tool to cause the blade to cut the plaque; and
removing the plaque from the arterial wall.
